(19) United States
(12) Patent Application Publication
(10) Pub. No.: US 2011/0309267 A1
(43) Pub. Date: Dec. 22, 2011

(54) ITERATIVE TIME-REVERSAL ENHANCED TRANSMISSION SOLVING APPROACH

(75) Inventors: Meng Cui, Ashburn, VA (US); Changhuei Yang, Pasadena, CA (US)

(73) Assignee: CALIFORNIA INSTITUTE OF TECHNOLOGY, Pasadena, CA (US)

(21) Appl. No.: 13/157,194

(22) Filed: Jun. 9, 2011

Related U.S. Application Data
(60) Provisional application No. 61/355,326, filed on Jun. 16, 2010.

(51) Int. Cl. A61N 5/00

(52) U.S. Cl. 250/492.1

(57) ABSTRACT

A method, apparatus, and article of manufacture for irradiating a sample with electromagnetic (EM) radiation. A number of passes of EM radiation through a sample are formed and/or selected, wherein the EM radiation in each of the passes comprises (1) input EM radiation incident on the sample, and (2) transmitted EM radiation exiting the sample formed from the input EM radiation that is transmitted through the sample. A phase conjugate of the transmitted EM radiation is used as the input EM radiation in a next pass of the EM radiation. The number of passes results in one or more EM fields of the input EM radiation having at least a threshold transmittance through the sample.

### CROSS-REFERENCE TO RELATED APPLICATIONS

This application claims the benefit under 35 U.S.C. Section 119(e) of the following co-pending and commonly-assigned U.S. provisional patent application(s), which is/are incorporated by reference herein:

Provisional Application Ser. No. 61/355,326, filed on Jun. 16, 2010, by Meng Cui, Ying Min Wang, and Changhuei Yang, entitled “ITERATIVE TIME-REVERSAL ENHANCED TRANSMISSION SOLVING APPROACH,” attorneys' docket number 176.64-US-PI (CIT-5625-P).

This application is related to the following co-pending and commonly-assigned patent applications, which applications are incorporated by reference herein:

1. U.S. patent application Ser. No. 12/943,857, filed on Nov. 10, 2010, by Changhuei Yang and Meng Cui, entitled “TURBIDITY SUPPRESSION BY OPTICAL PHASE CONJUGATION USING A SPATIAL LIGHT MODULATOR,” attorneys' docket number 176.58-US-U1, which application claims the benefit under 35 U.S.C. §119(e) of the following co-pending and commonly-assigned U.S. provisional patent applications, which are incorporated by reference herein:

Provisional Application Ser. No. 61/259,975, filed on Nov. 10, 2009, by Changhuei Yang and Meng Cui, entitled “APPROACHES FOR BUILDING COMPACT FLUORESCENCE MICROSCOPES,” attorneys' docket number 176.58-US-P1 (CIT-5473-P1);

Provisional Application Ser. No. 61/260,316, filed on Nov. 11, 2009, by Changhuei Yang and Meng Cui, entitled “APPLICATIONS OF TURBIDITY SUPPRESSION BY OPTICAL PHASE CONJUGATION,” attorneys' docket number 176.58-US-P2 (CIT-5473-P2);

Provisional Patent Application Ser. No. 61/376,202, filed on Aug. 23, 2010, by Meng Cui and Changhuei Yang, entitled “OPTICAL PHASE CONJUGATION 4PI MICROSCOPE,” attorneys' docket no. 176.60-US-PI (CIT-5663-P); and

Provisional Application Ser. No. 61/355,328, filed on Jun. 16, 2010 by Meng Cui, Ying Min Wang and Changhuei Yang, entitled “ACOUSTIC ASSISTED PHASE CONJUGATE OPTICAL TOMOGRAPHY,” attorneys' docket number 176.59-US-P1 (CIT-5626-P);

2. U.S. Utility patent application Ser. No. 12/886,320, filed on Sep. 20, 2010, by Zahid Yaqoob, Emily McDowell and Changhuei Yang, entitled “OPTICAL PHASE PROCESSING IN A SCATTERING MEDIUM,” attorney's docket number 176.54-US-D1, which application is a divisional of U.S. Utility patent application Ser. No. 11/868,394, filed on Oct. 5, 2007, by Zahid Yaqoob, Emily McDowell and Changhuei Yang, entitled “TURBIDITY ELIMINATION USING OPTICAL PHASE CONJUGATION AND ITS APPLICATIONS,” attorney's docket number 176.54-US-U1, which application claims priority under 35 U.S.C. §119(e) to commonly-assigned U.S. Provisional Patent Application Ser. No. 60/850,356, filed on Oct. 6, 2006, by Zahid Yaqoob, Emily McDowell and Changhuei Yang, entitled “TURBIDITY ELIMINATION USING OPTICAL PHASE CONJUGATION AND ITS APPLICATIONS,” attorney's docket number 176.54-US-P1;

3. U.S. Utility application Ser. No. 12/943,841, filed on Nov. 10, 2010, by Meng Cui, Ying Min Wang, Changhuei Yang and Charles DiMarzio, entitled “ACOUSTIC ASSISTED PHASE CONJUGATE OPTICAL TOMOGRAPHY,” attorney's docket number 176.59-US-U1, which application claims priority under 35 U.S.C. §119(e) to co-pending and commonly-assigned U.S. Provisional Application Ser. No. 61/355,328, filed on Jun. 16, 2010, by Meng Cui, Ying Min Wang, and Changhuei Yang, entitled “ACOUSTIC ASSISTED PHASE CONJUGATE OPTICAL TOMOGRAPHY,” attorney's docket number 176.59-US-PI (CIT-5626-P); U.S. Provisional Application Ser. No. 61/259,975, filed on Nov. 10, 2009, by Changhuei Yang and Meng Cui, entitled “APPROACHES FOR BUILDING COMPACT FLUORESCENCE MICROSCOPES,” attorneys' docket number 176.58-US-P1 (CIT-5473-P1); U.S. Provisional Application Ser. No. 61/260,316, filed on Nov. 11, 2009, by Changhuei Yang and Meng Cui, entitled “APPLICATIONS OF TURBIDITY SUPPRESSION BY OPTICAL PHASE CONJUGATION,” attorneys' docket number 176.58-US-P2 (CIT-5473-P2); and U.S. Provisional Patent Application Ser. No. 61/376,202, filed on Aug. 23, 2010, by Meng Cui and Changhuei Yang, entitled “OPTICAL PHASE CONJUGATION 4PI MICROSCOPE,” attorneys' docket no. 176.60-US-P1 (CIT-5663-P); and

4. U.S. Utility application Ser. No. 12/943,818, filed on Nov. 10, 2010, by Meng Cui and Changhuei Yang, entitled “OPTICAL PHASE CONJUGATION 4PI MICROSCOPE,” attorney's docket number 176.60-US-U1, which application claims priority under 35 U.S.C. §119(e) to co-pending and commonly-assigned U.S. Provisional Application Ser. No. 61/376,202, filed on Aug. 23, 2010, by Meng Cui and Changhuei Yang, entitled “OPTICAL PHASE CONJUGATION 4PI MICROSCOPE,” attorney's docket number 176.60-US-PI (CIT-5663-P); U.S. Provisional Application Ser. No. 61/259,975, filed on Nov. 10, 2009, by Changhuei Yang and Meng Cui, entitled “APPROACHES FOR BUILDING COMPACT FLUORESCENCE MICROSCOPES,” attorneys' docket number 176.58-US-P1 (CIT-5473-P1); U.S. Provisional Application Ser. No. 61/260,316, filed on Nov. 11, 2009, by Changhuei Yang and Meng Cui, entitled “APPLICATIONS OF TURBIDITY SUPPRESSION BY OPTICAL PHASE CONJUGATION,” attorneys' docket number 176.58-US-P2 (CIT-5473-P2); and U.S. Provisional Application Ser. No. 61/355,328, filed on Jun. 16, 2010 by Meng Cui, Ying Min Wang and Changhuei Yang, entitled “ACOUSTIC ASSISTED PHASE CONJUGATE OPTICAL TOMOGRAPHY,” attorneys' docket number 176.59-US-P1 (CIT-5626-P).

### STATEMENT REGARDING FEDERALLY SPONSORED RESEARCH AND DEVELOPMENT

The invention was made with government support under Grant No. EB008866 awarded by the National Institutes of Health. The government has certain rights in the invention.

### BACKGROUND OF THE INVENTION

1. Field of the Invention

Embodiments of the present invention relate generally to enhanced transmission through samples and photodynamic therapy, and in particular, to methods, apparatus, and article(s) of manufacture for enhancing transmission through samples and performing photodynamic therapy.

2. Description of the Related Art

(Note: This application references a number of different publications as indicated throughout the specification by reference numbers enclosed in brackets, e.g., [x]. A list of these different publications ordered according to these reference numbers can be found below in the section entitled “References.” Each of these publications is incorporated by reference herein.)

Radiation therapy, which is defined as the use of directed x-ray or gamma radiation to selectively target and kill tumor cells, is an important clinical cancer treatment option [1] (one or more embodiments of the present invention draw a distinction between radiation therapy and radioisotope therapy, which is useful for treating a smaller class of cancers). The effectiveness of such radiation in penetrating tissues and directly killing tumor cells are two key advantages. However, such radiation can also kill healthy cells along its trajectory.

Photodynamic therapy (PDT) [2, 3], which works by using light to activate photosensitive agents that preferentially accumulate in tumor cells, can provide greater discrimination ability. Photodynamic therapy offers other advantages, such as 1) use of safer radiation source(s)—optical radiation (especially in the 600 nm range) is far less mutagenic, if at all, and 2) requiring co-localization of both photosensitive agent and optical radiation to kill cells—which means that other organs that can accumulate the agent can be spared damage by minimizing their exposure to the radiation. Unfortunately, PDT has a key limitation that restricts its application scope. Specifically, photodynamic therapy is only effective to ˜1 cm depth of the illuminated tissue.

This limitation is imposed by the extreme turbidity or scattering exhibited by biological tissues in the optical regime. As a point of reference, the mean scattering length of 630 nm light in dermis is 50 microns [4]. In very much the same way that a car's headlights cannot penetrate well through thick fog, tissue turbidity prevents effective light delivery through tissues by scattering and diverting light from its forward trajectory.

The limited depth penetration issue impacts on the utility of PDT in two major ways. First, it implies that a much higher incident fluence on the tissue surface is required in order to ensure that sufficient light is able to make its way deeper into the tissue to activate the PDT agents. Therefore, the PDT treatment is a delicate balancing act—the clinician needs to make sure enough light gets through, and he/she needs to make sure the amount of light used is below the threshold for superficial tissue damage. Second, the limited penetration implies that PDT is not appropriate for more advanced stage cancer treatment where the tumor may be more extensive and more deeply rooted. In fact, the recurrence rate for PDT treatment is relatively high [5] and is likely related to this reason.

Interestingly, optimally delivering light through a scattering medium is not an impossible proposition. Simplistically speaking, if one has full knowledge of the positions and scattering profile of the scattering sites within the scattering medium, it would be possible to tailor the wavefront of an incident light field to optimally couple light to any specific point in the tissue. This approach capitalizes on the fact that scattering is a deterministic process. Such an idea has recently been explored by other groups for very simple and non-biological scattering media. Unfortunately, the high complexity of typical tissues has prevented the ability to fully characterize the tissue with sufficient detail, and within an adequately short time frame to accomplish such wavefront tailoring. One or more embodiments of the present invention solve these problems.

### SUMMARY OF THE INVENTION

Tissue turbidity in the optical regime is a very significant problem in general and is the key obstacle that prevents deep tissue imaging and therapy. One or more embodiments of the present invention tackle one or more consequences of tissue turbidity, via a fundamentally different and much more direct technological approach than prior works.

One or more embodiments of the present invention model the input and output face of a tissue as comprising of a set of ‘open’, ‘close’ and ‘semi-close’ channels [13]. Under normal conditions, prior to the present invention, light was sub-optimally delivered through tissue by applying optical power to each of these channels in a stochastically even fashion.

One or more embodiments of the present invention implement time-reversal tissue scattering suppression via optical phase conjugation, using fast and efficient wavefront tailoring technology, to enhance light delivery through tissues.

One or more embodiments disclose a method for irradiating a sample with electromagnetic (EM) radiation, comprising irradiating a sample with the EM radiation, wherein: (i) the EM radiation comprises one or more EM fields having at least a threshold transmittance or threshold scattering amount through the sample, (ii) the threshold transmittance or threshold scattering amount results from the EM radiation having made a threshold number of passes through the sample, (iii) the EM radiation in each of the passes comprises (1) input EM radiation incident on the sample, and (2) transmitted EM radiation exiting the sample formed from the input EM radiation that is transmitted through the sample, and (iv) a phase conjugate of the transmitted EM radiation is used as the input EM radiation in a next pass of the EM radiation.

In one or more embodiments, the number of passes is such that the EM fields have maximum transmittance through the sample.

In one or more embodiments, the threshold transmittance is such that a transmission of the EM radiation through one or more most transmissive channels of the sample is increased by a threshold amount as compared to a transmission through one or more less transmissive channels of the sample. For example, the method may select a number n of the passes such that a transmission or transmittance of the EM radiation through the sample at the n^{th} pass does not change by more than 10% as compared to a transmission of the EM radiation through the sample in an immediately preceding pass. For example, the threshold transmittance may be such that λ₁/λ₂)ⁿ≧5, wherein λ₁ is a measure of a transmittance of a most transmissive of the channels, λ₂ is a measure of the transmittance of a next most transmissive of the channels, and n is the number of passes of the EM radiation through the sample.

In one or more embodiments, the number of passes does not depend on the threshold transmittivity if two or three of the most transmissive channels have transmittivities that are at least twice a mean value of the transmittivity of all the channels, or a group of the channels have one or more transmittivities that are similar (e.g., within 5% of each other).

One or more embodiments maintain or increase a power of the input EM radiation in one or more of the passes as compared to a power of the input EM radiation in a previous pass of the EM radiation.

One or more embodiments repeat steps, thereby continuously re-optimizing and compensating for scatterers in the sample shifting over time. For example, repeating steps may update the number of passes and/or maintain or increase the threshold transmittance as a function of time. In one or more embodiments, the number of passes depends on feedback from a result of the irradiating step.

One or more embodiments further disclose an apparatus to perform the steps. In one or more embodiments, the sample is tissue positioned between a first phase conjugator and a second phase conjugator, wherein the first phase conjugator produces the phase conjugate of the transmitted radiation from the even numbered passes, the second phase conjugator produces the phase conjugate of the transmitted radiation from the odd numbered passes, the EM radiation comprises one or more optical or near infrared wavelengths, and the number of passes are formed within a scattering time of the tissue. One or more embodiments may use one or more spatial light modulators to form the phase conjugate of the transmitted light.

One or more embodiments of the present invention quickly converge on a wavefront solution that optimally couples into one of the ‘open’ channels and then employs this wavefront to enhance light delivery through the tissue in question. One or more embodiments of the convergence approach use a time-reversal optoelectronic method, termed digital optical phase conjugation (DOPC), to repeatedly bounce time-reversed light field through the target to preferentially elicit the optimal open channel solution. The proposed approach is highly novel and the DOPC system invention is one of the key enablers.

One or more embodiments provide a computer readable storage medium encoded with computer program instructions which when accessed by a computer cause the computer to load the program instructions to a memory therein creating a special purpose data structure causing the computer to operate as a specially programmed computer, executing a method of irradiating a sample with electromagnetic (EM) radiation, comprising receiving, in the specially programmed computer, values for one or more electromagnetic (EM) fields at an input face and an output face of the sample, for one or more passes of the EM fields through the sample; using the EM fields to obtain, in the specially programmed computer, one or more transmittivities of the sample for the one or more passes; comparing, in the specially programmed computer, the transmittivities with a threshold transmittance that is acceptable for the application; and selecting, in the specially programmed computer, the number of the passes of the EM fields that obtains the threshold transmittance and that is outputted from the computer and used to irradiate the sample.

One or more embodiments of the present invention describe implementations of this technology to enhance light transmission through tissues, study the efficacy of this technology to improve PDT in various tissues, and improve PDT. For example, one or more embodiments may perform photodynamic therapy, wherein the target comprises one or more photosensitive agents (e.g., at a depth of more than 1 cm below a surface of the sample), and the irradiating of the photosensitive agents triggers the photodynamic therapy.

One or more embodiments of the present invention may at least boost light delivery to a depth of 1cm inside tissue with at least a 10-fold improvement, for a wavelength 630 nm. One or more embodiments may accomplish a depth penetration through tissue of at least 6 cm.

Embodiments of the present invention may open up major new biophotonics areas, with significant applications in biomedical imaging, and provide therapeutic benefits that were previously unobtainable. However, it is not intended that embodiments of the present invention are limited to particular applications.

### BRIEF DESCRIPTION OF THE DRAWINGS

Referring now to the drawings in which like reference numbers represent corresponding parts throughout:

FIG. 1(a) illustrates a cross-section of a scattering medium and how light transmission through a scattering medium can be interpreted as a summation of light fields travelling through orthogonal channel modes, according to one or more embodiments of the present invention;

FIG. 1(b) illustrates how an approach for finding the optimal wavefront, that couples to the maximally open channel, comprises repeatedly time-reversing the transmission light field through the medium until convergence occurs, according to one or more embodiments of the present invention;

FIG. 2 illustrates a Digital Optical Phase Conjugation (DOPC) System as used in one or more embodiments of the present invention;

FIG. 3 illustrates an apparatus comprising two DOPC units coupled to opposing sides of a target or sample, for implementing the iterative time-reversal enhanced transmission solving approach, according to one or more embodiments of the present invention;

FIG. 4 illustrates a method for performing photodynamic therapy (PDT), according to one or more embodiments of the present invention;

FIG. 5 illustrates a geometry, for performing photodynamic therapy (PDT), according to one or more embodiments of the present invention;

FIG. 6 illustrates a method for obtaining an electromagnetic (EM) field having increased transmission through a sample, according to one or more embodiments of the present invention;

FIG. 7 transmission through a sample, according to one or more embodiments of the present invention;

FIG. 8 illustrates an apparatus for obtaining an electromagnetic (EM) field having increased transmission through a sample, according to one or more embodiments of the present invention;

FIG. 9 is an exemplary hardware and software environment used to implement one or more embodiments of the invention; and

FIG. 10 schematically illustrates a typical distributed computer system using a network to connect client computers to server computers, according to one or more embodiments of the present invention.

### DETAILED DESCRIPTION OF THE PREFERRED EMBODIMENTS

In the following description, reference is made to the accompanying drawings which form a part hereof, and which is shown, by way of illustration, several embodiments of the present invention. It is understood that other embodiments may be utilized and structural changes may be made without departing from the scope of the present invention.

Overview

The description provided herein illustrates that photodynamic therapy (PDT), which works by using light to activate photosensitive agents that preferentially accumulate in tumors, is a potentially versatile cancer therapy method. Its advantages include: 1) the use of safer radiation source(s)—optical radiation (especially in the 600 nm range) has little mutagenic potentials, and 2) requiring co-localization of both photosensitive agent and optical radiation to kill cells—which means that other organs that can accumulate the agent can be spared damage by minimizing their exposure to the radiation. Unfortunately, until now, PDT had a key limitation that restricted its application scope—it was only effective to ˜1 cm depth of the illuminated tissue.

This limitation is imposed by the extreme turbidity, or scattering, exhibited by biological tissues in the optical regime. In very much the same way that a car's headlights cannot penetrate well through thick fog, tissue turbidity prevents effective light transmission by scattering and diverting light from its forward trajectory.

The limited depth penetration issue impacts on the utility of PDT in two major ways. First, it implies that a much higher incident fluence on the tissue surface is required in order to ensure that sufficient light is able to make its way deeper into the tissue to activate the PDT agents. Therefore, the PDT treatment is a delicate balancing act—the clinician needs to make sure enough light gets through, and he/she needs to make sure the amount of light used is below the threshold for superficial tissue damage. Second, the limited penetration implies that PDT is not appropriate for more advanced stage cancer treatment where the tumor may be more extensive and more deeply rooted. In fact, the relapse rate for PDT treatment is relatively high and is likely related to this reason—PDT simply isn't able to completely eliminate trace amounts of deep tumor cells.

The description presented herein illustrates that optimally delivering light through a scattering medium, such as tissues, is not an impossible proposition. In fact, scattering is actually a deterministic process and there exists an optimal wavefront solution that can maximally transmit light through a scattering medium via an ‘open’ channel mode of the medium. Finding this mode is equivalent to solving for the strongest singular value decomposition eigenmode of the medium's transfer matrix. Unfortunately, the sheer size of the transfer matrix for a complex scattering medium, such as tissue, makes numerically solving for the solution highly impractical.

One or more embodiments of the present invention use time-reversal based scattering suppression via optical phase conjugation to provide a much quicker and practical way to tackle this problem. In addition, one or more embodiments of the present invention use a versatile optoelectronic system that generates a time-reversed light field effectively and quickly. By repeatedly time-reversing a seed light field back-and-forth through the target medium, quick convergence on the correct wavefront for maximal light transmission may be achieved.

This approach is unique, highly novel and highly suited to address PDT's improved penetration needs.

Applications of one or more embodiments of the present invention include enhancing light transmission through samples (e.g., tissues) and/or improving PDT of the sample (e.g., tissues). One or more embodiments may use an animal model as the sample. One or more embodiments may boost light delivery through a tissue sample, for a wavelength 630 nm, to a depth of 1 cm with at least a 10-fold improvement. In addition, one or more embodiments may enable light to penetrate to a depth of at least 6 cm through tissue. One or more embodiments may also enhance light transmission through a living tumor (e.g., mouse tumor) for PDT, and deliver PDT action into a tissue thickness of ˜1 cm, or provide enhanced delivery for PDT treatment through at least a 6 cm thick tissue thickness. However, these thicknesses are purely provided as examples and the benefits of one or more embodiments of the present invention are not limited to particular tissue thicknesses.

Mathematical Model

To clearly understand one or more embodiments of the present invention, it is fruitful to express the issue of scattering medium light transmission mathematically. The transmission of light through a scattering medium can be expressed as a transfer matrix as follows:

[E_{out,face2}]=[K][E_{in,face1}] Eq.(1)

[E_{out,face1}]=[K]^{T}[E_{in,face2}] Eq.(2)

where [E_{in,face1}], [E_{in,face 2}], [E_{out,face1}], and [E_{out,face1}] are Nx1 column vectors representing the input or output light field at either face 1 or face 2 of the sample, and N equals the number of addressable/addressed/sample points on the face [14]. [K] is the transfer matrix (transmission matrix) that links the light field amplitude on face 1 to face 2 of the medium; and T denotes transpose.

The above model can be applied to the case where a spatial light modulator (SLM) or photorefractive crystal is used to create a specific wavefront incident on the sample. The amplitude and phase of the light field at each given point on a given area of the input face (the area does not have to cover the entire face) is given by an element in [E_{in,face1}]. One or more embodiments of the present invention characterize the transmitted wavefront by making wavefront amplitude and phase point measurements at each point on a given area of the output face (the area does not have to cover entire face). Each point on the input and/or output faces of the sample, where amplitude and phase measurements are performed, may be an addressed point corresponding to speckle grains as described in [14].

The transfer matrix can be re-expressed as a singular value decomposition (SVD) matrix set:

$$\begin{matrix}[ K ] = U Λ V \\ = U [ \begin{matrix}{λ}_{1} & & & \\ & {λ}_{2} & & \\ & & ⋱ & \\ & & & {λ}_{N}\end{matrix} ] V \\ = U [ \begin{matrix}{λ}_{1} & & & \\ & {λ}_{2} & & \\ & & mostly close to 0 & \\ & & & \sim 0\end{matrix} ] V\end{matrix} \qquad Eq . ( 3 )$$

where U and V are unitary matrices, Λ is a diagonal matrix containing the eigenvalues, and the matrix Λ is arranged so that λ₁>λ₂>λ₃> . . . λᵢ . . . >λ_{N}, for 1<i≦N, where N corresponds to the number of addressable/sampled points [14] on the sample and transmission channels involved. In the case of a sufficiently thick scattering medium, most of the eigenvalues λᵢ are close to 0 (corresponding to ‘closed’ channels) and only the top few in the top left portion of the diagonal are sufficiently significant (corresponding to ‘open’ channels). λᵢ is a measure of the transmittivity (or transmittance) of the channel i. λᵢ has a maximum value of 1, and channels with value 1 correspond to fully open channels that can transmit light without loss. In some examples, an open channel may be considered to have a transmittance greater than 0.8.

In some examples, transmittivitty/transmittance may be obtained by dividing intensity of light exiting the sample at the output face, by the intensity of light inputted at the input face. The transmittivity/transmittance may also be measured for one or more channels, and for one or more passes of light through the sample, for example.

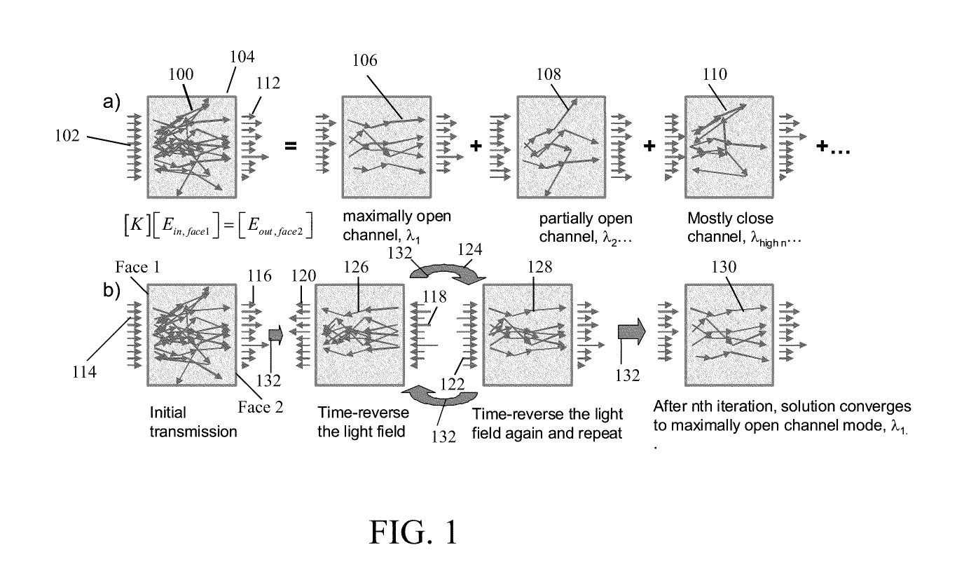

FIG. 1(a) illustrates how light transmission 100 of an input light 102 through a scattering medium 104 can be interpreted as a summation (indicated as + in FIG. 1(a)) of light fields travelling 106, 108, 110 through orthogonal channel modes to produce output light 112. FIG. 1(a) illustrates the light transmission 100 comprises light 106 travelling through a maximally open channel λ₁, light 108 travelling through a partially open channel 22, and the light 110 travelling through a mostly closed channel λᵢ, where n is high (n is the number of passes).

From the set of equations Eq. (1), Eq. (2), and Eq. (3), the inventors note that randomly choosing a light field wavefront [E_{in,face1}] would indiscriminately channel power through all modes, and only the power that goes through the open channels would contribute to [E_{out,face2}] significantly.

Instead, one or more embodiments of the present invention choose [E_{in,face2}] so that it is the right eigenvector of [K] associated with eigenvalue λ₁, so that much more power is channeled through the scattering medium and creates a much stronger transmission. Accordingly, this solution (Electric field with eigenvalue λ₁) enables enhanced light transmission for PDT applications.

While mathematically easy to express, finding such an optimal wavefront by measuring and solving [K] is very challenging experimentally, especially if N is large, such as N=2.6×10⁵ (N is the number of addressed/sampled points on the faces of the sample where field amplitude and phase measurements are performed, and corresponds to the number of transmission channels i through the sample that are used, and is proportional to the area of the sample that is illuminated and through which turbidity suppression is to be performed. If N=2.6×10⁵, a full characterization approach would require far too many measurements (N²=7×10¹⁰ elements in [K]).

Solution Method

Fortunately, one or more embodiments of the present invention use a quick way to find the optimal [E_{in,face1}] without actually quantifying [K] (a very large matrix). It is noted that scattering is a deterministic process, and that a time-reversed light field can undo the effects of scattering. Specifically, the optimal [E_{in,face1}] may be found by recording the phase and amplitude of the propagating scattered light field and reproducing a back propagating optical phase conjugate (OPC) or time-reversed field. This field retraces its trajectory through the scattering medium and returns the original input light field (minus loss from closed channels).

An OPC field is simply a copy of the transmitted light field with the signs of the phase term reversed. In the formalism of Eq. (1) and Eq. (2), this is given by [E_{out,face2}]*, where * denotes the field is a conjugate. Such an OPC field may be generated by holography or optoelectronically, as the inventors have demonstrated [9]. The optoelectronic approach may be particularly advantageous because it actually creates an OPC copy of the light field from a secondary ‘blank’ light beam, rather than ‘reflecting’ the original transmission. This allows the total power of each OPC playback to be boosted to compensate for transmission loss.

To find the optimal [E_{in,face1}] for maximum transmission, one or more embodiments of the present invention may employ the scheme shown in FIG. 1(b).

The scheme comprises (a) sending a uniform light field 114 onto face 1 of the sample and measuring/detecting the transmission of the input field 114 through the sample emerging as transmitted light field 116 (and, optionally, measuring the amount of transmission of the input light field 114 through the sample); (b) then sending an OPC or time-reversed copy 118 of the transmitted light field 116 back through face 2 and measuring/detecting the transmission of the OPC field 118 emerging as transmitted light field 120 at face 1 (and, optionally, measuring the amount of transmission of the OPC field 118 through the sample), and (c) then sending an OPC copy 122 of the transmitted light field 120 back through face 1 and repeating 124 steps (b) and (c). In this way, the wavefront incident on faces 1 and 2 of the sample is patterned to couple optimally to (and travel 126, 128 through) the maximally open channel (which has an eigenvalue λ₁). Eventually, after an n^{th} iteration, the solution converges to a maximally open channel mode λ₁ and [E_{out,face1}]*_{nth pass} converges to equal the light field 130 having the optimal wavefront for maximum transmission. Having fully characterized this optimal wavefront, a strong light field having the optimal wavefront for maximum transmission can be sent through the tissue in the maximally open channel mode λ₁, e.g., for PDT applications. The sequence of steps in FIG. 1(b) is denoted by arrows 132.

The inventors note the process can be well understood by noting that the power sent by the initial light field 114 into the sample's 104 open channels transmits 126 through the medium 104 with greater efficiency than those sent through the closed or near-closed channels. By sending a time-reversed copy 118 of the light field back, the process forces the light to retrace their paths through the channels and again enhance the net transmittivities of the open channels versus the closed or near-closed ones. In each cycle, the process further distills the light field corresponding to the optimal open channel (eigenvalue λ₁). If the ratio of λ₂/λ₁=0.9 or less, it may take 22 passes to achieve convergence. If there is a group of λ₁, λ₂, . . . λᵢ . . . λ_{N} that are close in values (e.g., within 5% of each other), one or more embodiments may consider that stopping the iteration at 22 passes still achieves a near optimal transmission solution (because, according to these embodiments, since the λᵢ are all close in value to λ₁, they are all good open channels).

Mathematically, the convergence on the optimal solution may be expressed as:)

[E_{out,face1}]*_{nth pass}=([K]^{†}[K])^{n/2}[E_{in,face1}]=V^{†}Λ*^{n/2}Λ^{n/2}V[E_{in,face1]=const*(}1st row of V^{†})≈[E_{in,face1}]_{optimal transmission} Eq.(4)

where n is the number of passes of the electric field or light through the sample and the inventors note that the 1^{st} column of V and the 1^{st} row of V^{†} is the right SVD eigenvector corresponding to λ₁, the best open channel.

Findings on Turbidity Suppression by Optical Phase Conjugation (OPC) OPC Through Thick Tissue Sections

While using a time-reversed light field to undo the effects of scattering has been demonstrated to work with distorted glass plates [15], one of the inventors led a group that was the first to adapt the concept to suppress tissue scattering [16].

OPC's ability to undo scattering through tissue sections up to 6 mm thick at a wavelength of 532 nm has been demonstrated [12]. In addition, the tissue scattering coefficient for this tissue section was also measured to be 30.3 mm¹ at this 532 nm wavelength, in a separate measurement [12]. These results imply that, on average, a photon is scattered more than 300 times in the 1 cm thick tissue section used in these measurements. The thickness of the 1 cm section, and the size of the phase conjugate mirror (a photorefractive crystal for these measurements) also imply that only <0.02% of the available wavefront was recorded during the measurement. Yet, the time-reverse playback of this incomplete wavefront was still capable of scattering path retracing, albeit with a diminished efficiency [17]. These measurements demonstrate the robustness of the OPC phenomenon and OPC phenomenon's tolerance to noise.

OPC Through Living Tissues

One or more embodiments of invention may be applied to living tissues. In one example, it is required that the OPC process is stable when living tissues are involved. OPC has been performed through a scattering medium comprising a live rabbit ear [18] (a shaved ear, ˜1 mm thick, of a New Zealand rabbit). The measurements performed found that tissue movements do cause scatterer position shifts (corresponding to a [K] that is changing in time). However, an OPC signal that is never-the-less relatively stable over durations of ˜1.5 seconds (time constant) may be used. One or more embodiments perform the iteration procedure faster than ˜few seconds (faster than the time for scatterer position shifts) in order to create enhanced light transmission.

Apparatus: Digital Optical Phase Conjugation (DOPC)

One or more embodiments of the iterative method of solving for the optimal wavefront solution for enhanced light transmission depend on the ability to effectively generate an OPC or time-reversed copy of the light field. Simple back-and-forth reflections (from mirrors) will not lead to the open channel convergence solution.

The generation of OPC field traditionally relied on various nonlinear effects, such as the photorefractive effect or optical Kerr effect. Generally, the effective OPC reflectivity, defined as the power ratio of the generated OPC wave to the input signal, is fairly low (significantly less than unity).

However, one or more embodiments of the present invention use an OPC system that is capable of recording a weak light field, and that is capable of generating a strong time-reversed light field during the playback process, in order to avoid ending up with a very weak field after several iterations.

The inventors' DOPC technology is used in one or more embodiments of the present invention. The DOPC system may provide the advantage of creating the OPC copy from a separate ‘blank’ input beam, rather than attempting to ‘time-reverse reflect’ the actual light field. By increasing the ‘blank’ beam's power, one or more embodiments of the invention compensate for transmission loss incurred in the iteration process.

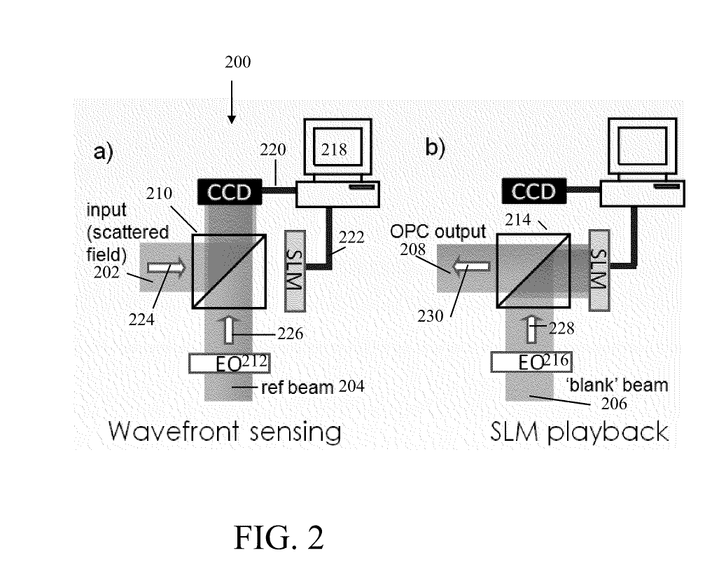

The inventors' all-optoelectronic system, with such a capability of recording a weak light field and generating a strong time reversed light field during playback [9], is a developed technology. The DOPC system may take two separate steps to generate the OPC fields, as shown in FIG. 2.

In step 1 (FIG. 2(a)), an interferometric phase stepping system 200 is used to perform wavefront sensing, by measuring the amplitude and phase variations of the input (a target light field 202). Specifically, a phase-stepping interference pattern resulting from interference of the reference beam 204 and the input (e.g., target light field or scattered field 202) is recorded on a Charge Coupled Display (CCD), and the amplitude and phase variations of the target light field 202 are determined from the recorded phase-stepping interference pattern.

In step 2 (FIG. 2(b)), a spatial light modulator (SLM) is used to perform SLM playback by modifying a ‘blank’ light field of a blank beam 206 into an appropriate OPC copy 208 (or OPC output) of the input scattered field 202. The OPC reflectivity from the SLM may be adjusted freely by changing the power of the input ‘blank’ light field 206. Also shown are a beamsplitter 210, for combining the input 202 and the reference beam 204 onto the CCD for interference, an electro-optic modulator (EO, 212) for modulating the reference beam 204 to create the interference of reference beam 204 and input 202, a beamsplitter 214 for directing the blank beam 206 onto the SLM and transmitting/directing the OPC copy 208, and an EO 216 for controlling the blank beam 206. Also shown is a computer 218 coupled 220,222 to the CCD and the SLM, for processing the interference pattern measured on the CCD and obtaining the electric field amplitude and phase of the input, and providing the SLM with the appropriate electric field amplitude and phase to produce the time reversed copy 208. Any type of computer or processor, such as (but not limited to) a mainframe, minicomputer, or personal computer, or computer configuration, such as a timesharing mainframe, local area network, or standalone personal computer, internet or web-based processing/server, could be used with the present invention. The arrows 224, 226, 228, and 230 indicate propagation direction of the input 202, reference beam 204, blank beam 206, and OPC output 208, respectively. The light 202, 204, 226, 228 comprising light fields may include beams of light, or collimated beams of light, etc.

Further information on the DOPC can be found in U.S. patent application Ser. No. 12/943,857, filed on Nov. 10, 2010, by Changhuei Yang and Meng Cui, entitled “TURBIDITY SUPPRESSION BY OPTICAL PHASE CONJUGATION USING A SPATIAL LIGHT MODULATOR,” attorneys' docket number 176.58-US-U1, which application is incorporated by reference herein.

One or more embodiments of the DOPC technology provide an appropriate optoelectronic OPC system that is well-suited for use in one or more embodiments of the present invention's enhanced light transmission system.

Enhancing Light Transmission Through Tissue and Tissue Phantoms

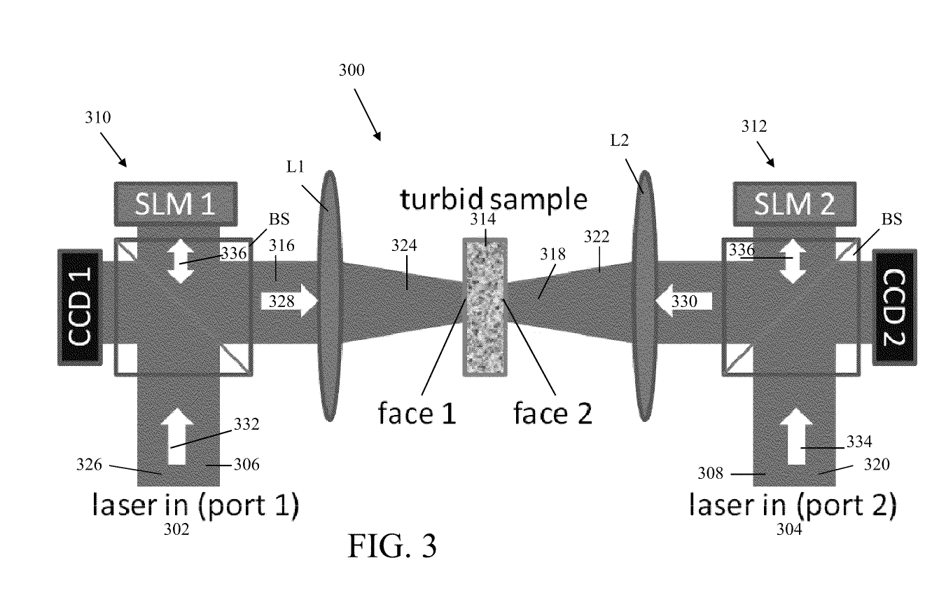

One or more embodiments of the present invention may enhance light transmission through tissue or tissue phantoms using the apparatus 300 shown in FIG. 3.

The apparatus 300 comprises two input ports, e.g., laser in (at port 1, 302) and laser in (at port 2 304), for receiving input light 306, 308. A light source (e.g., a modified Lasermate RML-635-X500 500 mW 630 nm laser, chosen for 630 nm center wavelength) may be used to provide the input light 306, 308.

The apparatus 300 further comprises two DOPC units 310, 312, wherein each DOPC unit 310,312 comprises an SLM and a CCD, wherein each DOPC unit 310, 312 faces opposing sides (face 1 and face 2) of the target 314 (e.g., turbid sample). The first DOPC unit 310 comprises a first CCD (CCD1) and a first SLM (SLM 1). The second DOPC unit 312 comprises a second CCD (CCD 2) and a second SLM (SLM 2)

During iteration (going from face 1 to face 2 of the sample 314), SLM 1 tailors the input laser wavefront of light 306 from port 1 302 into tailored light 316 and projects the tailored light 316 onto face 1. Next, at least part of the tailored light 316 is transmitted through the sample 314 and emerges as a transmitted light 318 emerging from face 2, wherein the transmitted light's 318 wavefront is interferometrically measured using the reference beam 320 (provided by the input laser light 308 through port 2) and CCD 2. Then, the SLM 2 tailors or creates a time-reversed copy 322 of the tailored light's 316 wavefront emerging from face 2 and projects the time reversed copy 322 onto face 2. At least part of the OPC copy 322 is transmitted through the sample 314, emerging as transmitted OPC light 324, and CCD 1 then measures the transmitted wavefront of transmitted OPC light 324 emerging from face 1. The process may be repeated iteratively until convergence occurs. The strength of the laser input 306, 308 provided through port 1 and port 2 (strong blank beam for playback and weak reference beam for wavefront measurement) may be adjusted depending on which DOPC unit 310, 312 is performing playback or recording at the time. The blank beam 326 and reference beam 320 may be included in the input light 306 and 308.

Also shown in FIG. 3 are beamsplitters BS allowing the light to pass onto the CCDs, SLMs, and the sample 314. Arrows 328, 330 indicate the direction of propagation of light 318 projected onto the sample 314, arrows 332, 334 indicate the propagation direction of input light 306, 308 from port 1 and port 2 respectively, and arrows 336 indicate the direction of propagation of the blank beam 326 inputted onto the SLM and the output OPC copy. Also shown are lenses L1 and L2 for imaging/focusing the light onto, and collecting light from, the sample 314.

A Basler A405KM CCD camera and a Boulder Nonlinear Systems XY Nematic 512 SLM may be used as the CCD and SLM in each DOPC unit 310, 312.

The tissue phantom 314 may comprise a collection of latex microspheres mixed in gelatin calibrated to provide comparable scattering characteristics as tissues.

The two DOPC units 310, 312 may be constructed and arranged so that they project onto the opposing faces of the tissue phantom 314 via relay optics.

Areas of interest on both faces may be 1 cm², for example (e.g., the light fields illuminate an area on the sample, or the transmission is enhanced for beam cross-section of, 1 cm²).

The transmission light field may be measured and recorded for a total of 50 iterations.

At each step, the net input field may be carefully boosted (e.g., by patterning the OPC copy on an input ‘blank’ laser beam, e.g., 306, 308 which can be adjusted in power) so that the net power delivered is fixed. By measuring the total transmission power, the transmission enhancement at each iteration may be quantified.

The transmission wavefront profiles may be examined and the speed at which the transmission wavefront profiles converge to the optimal solution may be noted. This speed may be used to determine how much the eigenvalues λₘ differs from each other.

One estimate is that convergence occurs at ˜22 iterations. However, convergence may be achieved faster or slower. In one example, it may be considered that more iterations are not required, because slower convergence may imply that there is an abundance of good open channels. Any superposition of the corresponding wavefronts may couple effectively and allow enhanced light transmission.

The system may be optimized to perform the iterations sufficiently quickly. The rate-limiting factor may be the frame rate of the SLM (70 Hz). The processing and playback may be optimized such that 22 iterations take less than 1 second to accomplish, for example.

The eigenvalues may follow an approximately exponential distribution [13]. For a sample that initially transmits at most only 0.2% of the light within the region of interest (approximately matching the transmission of the tissue phantom), transmission may be enhanced by a factor of at least 50.

The apparatus 300 may be used to study the amount of enhancement of light delivery, as a function of tissue thickness. To accomplish this, ever thicker pieces of tissue phantoms 314 may be employed, for example up to a thickness of at least 10 cm.

However, theoretical analysis does not indicate a mechanism by which this enhancement would start to fail. On the other hand, even if transmission improvements are present for all sample thickness, the net transmission for the thicker samples may simply be too weak to meaningfully activate the PDT agents. A limit of at least 1% of the total light transmitted via the enhanced transmission mode may be set as the threshold for acceptable light transmission.

Embodiments of the invention are not limited to a particular thickness of the tissue or tissue phantom, or through which light transmission is enhanced. Light transmission may be enhanced through a 1 cm tissue thickness (e.g., tissue phantom) by at least 10-fold, for example, and/or through at least a 6 cm thickness of tissue (e.g., tissue phantom).

Enhancing Light Transmission Through Living Tissue and Performing PDT

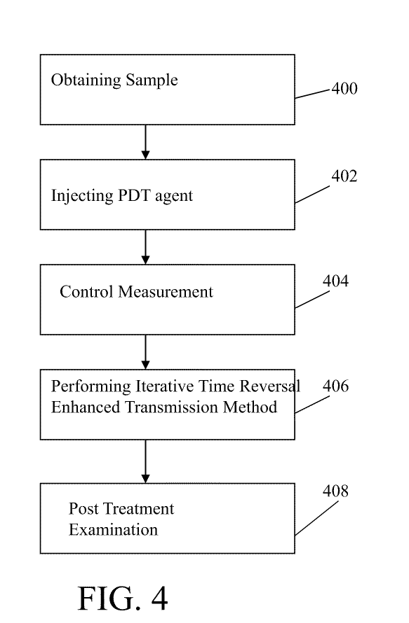

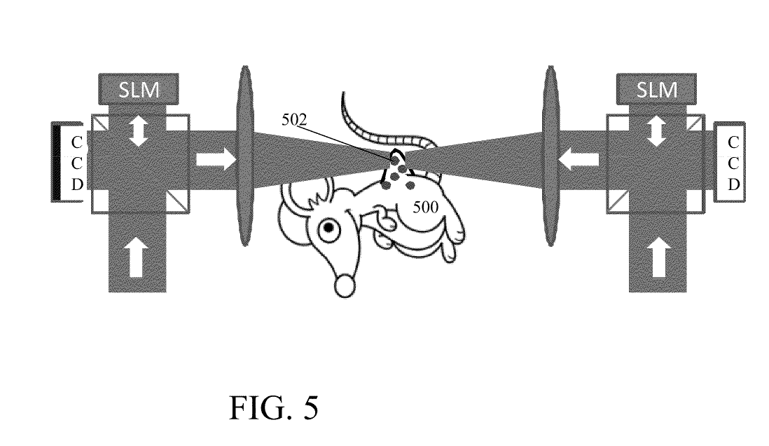

One or more embodiments of the present invention may enhance light transmission through a living tissue sample (e.g., but not limited to, a mouse tumor model or living mouse 400 tumor), for improving PDT efficiency, using the method of FIG. 4 and the apparatus of FIG. 5 (FIG. 5 uses the same method as illustrated as FIG. 3, except the sample 500 is a mouse, or living tissue, animal, human, or part thereof comprising one or more tumors 502). One or more embodiments deliver light for PDT action in a tissue thickness of at least ˜1 cm, for example.

Block 400 represents the step of obtaining a living sample comprising one or more (e.g., cancerous) tumors, for example. The tumors may be naturally occurring, or implanted and/or grown, in a mouse or other living organism. For example, the tumors 502 may be implanted and grown subcutaneously on nude mice (e.g., the tumors may be ˜1 cm in diameter). Alternatively, the tumor cells may be injected subcutaneously over the abdomen, in a location where the tumor is least likely to result in ambulatory difficulties as the tumor grows. In one example, when the tumors have exceeded 750 mg but not above 1500 mg in size, the animals may be ready for PDT.

Block 402 represents the step of injecting a PDT agent or photosensitizer into the living sample comprising the tumors 502. For example, Photofrin may be injected at a dosage of 2 mg/kg, followed by a waiting period of 40 hours to allow photofrin to preferentially accumulate in the tumor.

Block 404 represents the step of performing a control measurement on the living tissue sample comprising the PDT agent. The tumor 502 may be directly illuminated with a light dosage of e.g., 50 Joules/cm² at a wavelength 630 nm, setting the surface irradiance at 100 mW/cm² and an exposure time of ˜500 seconds. Multiple samples, e.g., 10 mice, may then be measured.

Block 406 represents the step of performing an iterative time-reversal enhanced transmission method (e.g., as illustrated in FIG. 3 or FIG. 1(b)), by illuminating the living sample with the light enhancement apparatus, e.g., the apparatus of FIG. 3 or FIG. 5. The tumor 502 location may be illuminated with the system of FIG. 3 or FIG. 5 using the same dosage as the control measurement of Block 404. The fluence may be kept intentionally below the generally prescribed dosage to allow an examination of the effectiveness of the PDT illumination strategy at low light fluence. Both the control and time-reversal enhanced transmission method may use the illumination geometry wherein illumination is transverse through the tumor 502. The time reversal enhanced transmission method of Block 406 may measure the same samples as in Block 404.

The system and method of FIG. 3, FIG. 5, or FIG. 1(b) applied to the living tissue may converge upon the optimal transmission wavefront within 1 second. Once convergence is reached, the converged solutions for the face-1-to-face-2 and the face-2-to-face-1 transmission may both be an eigenvector of the optimal open channel (one represents the right SVD eigenvector and the other the left SVD eigenvector). Both left-traveling and right-traveling converged wavefronts may equally transmit in an enhanced fashion. The system may be kept operating in iteration mode during the entire mouse or tissue illumination procedure, allowing the system 300 of FIG. 3 or FIG. 5 to continuously re-optimize the enhanced transmission solutions even as scatterers in the animal shift over time (and change [K]).

Block 408 represents post-treatment examination. In one example, the animals may be allowed to recover, and five days post treatment, half of the animals may be euthanized with pentobarbital (200 mg/kg, I.P.), and the other half may be euthanized ten days post treatment. The tumors may then be sectioned and measured for size. Pathology examination may quantify the extent of tumor reduction or increase.

Steps may be added or omitted, as desired.

Enhanced Delivery for PDT Treatment Through Thicker/More Complex Tissues

One or more embodiments of the present invention may enhance light transmission and/or perform PDT on a tumor that is sandwiched between two thick layers of normal tissues. This geometry may be simulated by placing a tumor mouse in contact with a normal mouse, such that the tumor is sandwiched between the two mouse bodies. For a mouse that is ˜2.5 cm wide, this provides a cushion of ˜2.5 cm of healthy tissues around a 1 cm width tumor, for example.

For the control measurement of the thicker, more complex samples (performed in Block 404), the target may be directly illuminated with a light dosage of 200 Joules/cm² at wavelength 630 nm. The illumination geometry may be through the first mouse body to the tumor and then through the second mouse body. The surface irradiance may be set at 200 mW/cm² with an exposure time of ˜1000 s. The same fluence in Blocks 406 and 404 may be employed.

In Block 406, the system or iterative method may be allowed to converge upon the optimal transmission wavefront. The convergence process may take longer for thicker/more complex scattering medium, e.g., a few seconds. During the initial part of the iteration process, the overall irradiance may be kept low (e.g., at 20 mW/cm² or 20 mW/cm² or less), so as to avoid unfairly illuminating the target with strong light field before convergence. Once convergence is reached, PDT treatment may begin by ramping up the irradiance to, e.g., 200 mW/cm² or 200 mW/cm² or less. The system may be kept operating in iteration mode during the entire tissue/mouse illumination procedure, so as to allow the system to continuously re-optimize the enhanced transmission solutions. The step of Block 408 may then be performed.

Thus, one or more embodiments of the present invention may enhance PDT action through deeper tissue (e.g., at least 6 cm thick) than is currently allowed by PDT procedure(s). However, embodiments of the present invention are not limited to particular sample or tissue thicknesses.

Process Steps

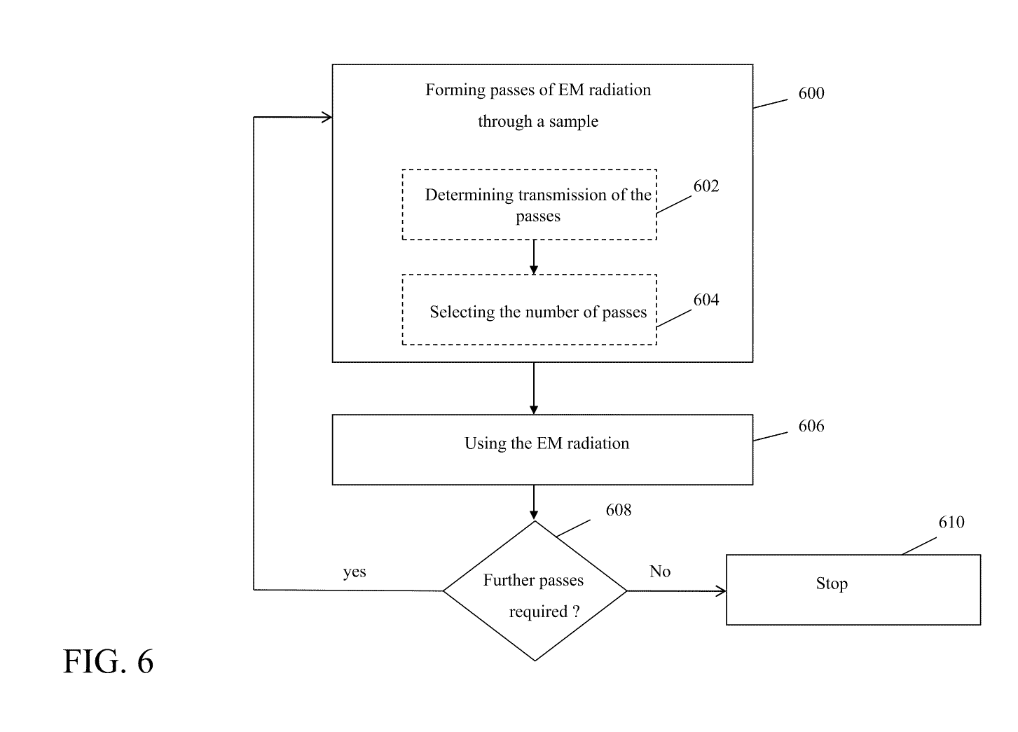

FIG. 6 illustrates a method for irradiating or illuminating a sample or specimen with electromagnetic (EM) radiation, according to one or more embodiments of the present invention. The method may obtain an electromagnetic (EM) field having increased transmission through a sample, iteratively solve for an optical field, tailor or filter an EM field, suppress, filter out, or eliminate unwanted transmissions or scattering pathways, or control transmittance of the sample. Further, the method may tailor the EM field for specific applications.

The method may comprise one or more of the following steps.

Block 600 represents forming and/or selecting and/or determining a number (0, 1, 2, a plurality, more than 1, a finite number, or infinite number, etc.) of passes of EM radiation (e.g., but not limited to, light, visible light, or comprising optical or near infrared wavelengths (e.g., 0.3 micrometers to 10 micrometers wavelength) through a sample, wherein (i) the EM radiation in each of the passes comprises (1) input EM radiation incident on the sample, and (2) transmitted EM radiation exiting the sample formed from the input EM radiation that is transmitted through the sample; (ii) a phase conjugate of the transmitted EM radiation is used as the input EM radiation in a next pass of the EM radiation; and (iii) the number of passes results in one or more EM fields of the EM radiation having at least a threshold transmittance or maximum threshold scattering amount through the sample (e.g., a maximum threshold scattering amount means the amount of scattering of the EM radiation by the sample has been reduced and has an upper bound, by eliminating reducing, or filtering out transmission of the EM radiation through less transmissive transmission channels of the sample or transmission channels of the sample that produce higher scattering of the EM radiation).

The sample may be tissue positioned between a first phase conjugator and a second phase conjugator (e.g., but not limited to, a holographic material, photorefractive crystal(s), such as lithium niobate, or DOPC units), wherein the first phase conjugator produces the phase conjugate of the transmitted radiation from the even numbered passes, the second phase conjugator produces the phase conjugate of the transmitted radiation from the odd numbered passes, and the EM radiation comprises one or more optical or near infrared wavelengths. The number of passes may be formed in a time that is less than the scattering time of the tissue or sample (e.g., less than ˜1 second).

The step may further comprise detecting the transmitted light on a sensor or detector to form a signal and using a spatial light modulator to form the phase conjugate of the transmitted light, using the signal. The sensor and the spatial light modulator may be such that convergence to an optimal electric field of the EM radiation through the sample that is animal or human tissue takes 1 second or less.

The method may tailor or produce an initial EM field/input EM radiation in the first pass based on, e.g., prior knowledge of the sample or other factors (e.g., prior iterations or instances of this method previously performed). For example, specific tailoring of the input light field may speed up convergence to the optimal field or reduce the number of passes required, or impart other desirable characteristics to the EM radiation. The tailoring may be performed by an SLM, for example. However, any light field may be used as the initially inputted light field, e.g., a plane wave may be used as the initially inputted light.

Block 602 represents determining the transmission of the sample. For example, the transmission/transmittance at one or more passes may be obtained by measuring the amount/intensity of input EM radiation inputted/incident onto (e.g., an area of the input face of) the sample, and measuring the amount/intensity of transmitted EM radiation exiting the sample formed from the input EM radiation that is transmitted through the sample. The amount/intensity of transmitted EM radiation in a pass may be compared to the amount/intensity of input EM radiation inputted onto the sample in that pass. The amount/intensity of transmitted EM radiation in a pass (e.g., the transmitted EM radiation detected on a detector after the pass) may be compared to the amount/intensity of transmitted EM radiation in one or more other passes (e.g., immediately preceding pass) to obtain a change in transmission. In other examples, the transmission, transmittance, or transmittivity of one or more transmission channels (e.g., open, closed, or partially open channels), at one or more of the passes, e.g., may also be obtained, for example, by calculation or measurement, or from a source such as a database, for example. This step may be performed during step 600, for example.

The step may further determine the power of the EM radiation (e.g., input EM radiation) in one or more of the passes.

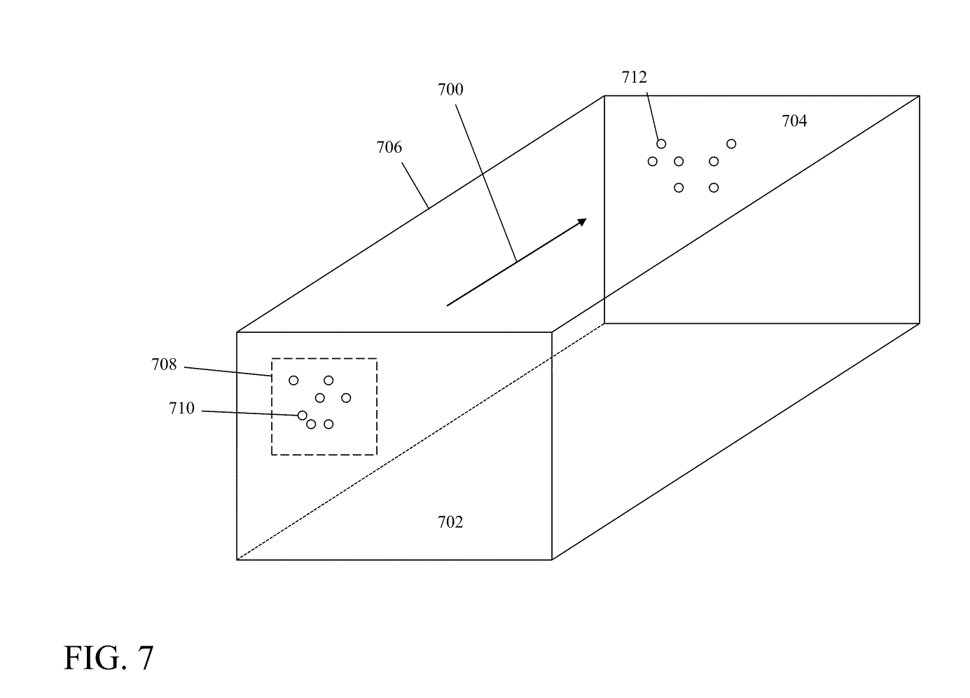

An exemplary illustration of one pass or transmission of EM radiation through a sample is shown in FIG. 7. FIG. 7 illustrates transmission pass of EM radiation 700, according to one or more embodiments of the present invention, from an input face 702 to an output face 704 (face 1 or face 2) of a sample 706. The transmitted EM radiation 700 is formed from input EM radiation incident on a selected irradiation area 708 of the sample 704. One or more addressable or measurement points within the selected irradiation area 708 are displayed and include input points 710 on the input face 702 where the input EM radiation can be measured. One or more addressable or measurement output points 712 on the output face 702 are also displayed, and include points 712 where the transmitted EM radiation exiting the sample, formed from the input EM radiation that is transmitted through the sample, may be measured.

For example, the transmittivities/transmittance may be given by measuring one or more electric field amplitudes and phases at one or more points 710, 712 on the input and output faces 702, 704 of the sample 706. For example, the electric field of the input EM radiation (incident on the sample) at one or more input points 710 on the input face 702 may be measured and squared to obtain the electric field squared at one or more input points 710, Eᵢₙₚᵤₜ². Eᵢₙₚᵤₜ² may then be summed over one or more of the input points 710 to obtain:

$${\sum }_{input \_ points} {E}_{input}^{2}$$

In addition, the electric field of the transmitted radiation (exiting the sample) at one or more output points 712 on the output face 704 may be measured and squared to obtain the electric field squared at one or more output points 712, Eₒᵤₜₚᵤₜ². Eₒᵤₜₚᵤₜ² may then be summed over the one or more of the output points 712 to obtain:

$${\sum }_{output \_ points} {E}_{output}^{2}$$

$$\frac{{\sum }_{output \_ points} {E}_{output}^{2}}{{\sum }_{input \_ points} {E}_{input}^{2}}$$

Then, dividing yields a quantity that can be used to obtain the transmission/transmittance for that pass. The transmittance may be obtained for one or more passes, and transmission/transmittance between the passes may be compared. For example, the transmittance of the EM radiation after the n^{th} pass may be enhanced or increased as compared to the transmittance of the EM radiation at one or more previous passes (including, e.g., the first pass).

The electric field may be represented by Ee^{iφ}, where E is the field amplitude and φ is the field phase

The intensity of the light/EM radiation, and the electric or EM fields may be determined using one or more detectors placed appropriately, e.g., CCDs in FIG. 5 or FIG. 3. In addition, it is not necessary to measure the amount/intensity of transmitted EM radiation, the output Electric field of the transmitted radiation, the amount/intensity of input EM radiation, or the input Electric field of the input EM radiation, at points 710/712 on the sample. For example, the amount/intensity of transmitted EM radiation, the output Electric field of the transmitted radiation, the amount/intensity of input EM radiation, or the input Electric field of the input EM radiation, may be measured at positions away from the sample 704, or by detectors positioned to collect/detect the transmitted EM radiation (e.g., collect/detect a whole or portion of a transmitted beam) or measure/detect the input EM radiation (e.g., collect/detect a whole or portion of an input beam).

In some embodiments, the EM fields detected on the detectors may be mapped to, or may be used to determine, Electric fields at the input and output points 710/712. In one embodiment, matrices for the electric/EM field, whose elements comprise the electric/EM fields at one or more of the points 710/712, may be constructed.

For some samples, selection of arbitrary single points 710, 712 on the input face and output faces may be almost guaranteed to hit a closed mode (the concept of open and close channel refers to the SVD matrix decomposition of the overall transmission matrix [21]). The transmission matrix may be given by the point-by-point transmission—specifically the (w,y) element of the transmission matrix is equal to the electric field transmission from point w on side A to point y on side B. However, in some examples, determining the matrices for the EM field, or determining the EM fields at the one or more points 710/712, or determining the transmission matrix, is not necessary. As noted above, one or more embodiments of the present invention use a quick way to find the optimal [E_{in,face1}] without actually quantifying the transmission matrix [K] or the electric field at each of the points 710/712.

Continuing with the steps of FIG. 6, Block 604 represents selecting/determining/controlling the number of passes that results in one or more electric fields of the EM radiation having the threshold transmittance or threshold scattering amount. This step may be performed before, after and/ or during block 600. The step may comprise tuning or adjusting one or more of a number of the passes and/or a power of the input EM radiation in one or more of the passes. The tuning/adjusting of the number of passes or power in the one or more passes may tailor an electric field of the phase conjugate EM radiation at a target within the sample so that the phase conjugate EM radiation images the target or causes therapy on or within the sample with an improvement as compared to a different number of the passes or a different power of the input EM radiation.

The step may comprise selecting a number n of the passes such that a transmission of the EM radiation at the n^{th} pass does not change by more than 10% as compared to a transmission of the EM radiation in an immediately preceding pass. The step may comprise selecting the number of passes wherein the electric field has reached a steady state solution where the electric field in the selected n^{th} pass has not changed (e.g., has not changed by more than 10% or 5%) as compared to the electric field in the immediately preceding pass. Any superposition of electric fields, wherein the electric fields comprise a superposition of the electric fields corresponding to a plurality of open channels, may be used. For example, the number of passes may be (e.g., selected/determined) such that the electric fields are described by a superposition of one or more modes (e.g., eigenmodes), wherein the modes/eigenmodes are eigenvectors of a singular matrix decomposition of the electric fields transmitted through the sample, and each of the eigenmodes represents one of the most/more open channels of the sample. For example, each of the eigenmodes may represent one of the three most open channels of the sample, and the electric field may be represented by a superposition of at most 3 eigenmodes representing the three most open channels of the sample. However, the superposition is not limited to a particular number of eigenmodes. Modes/Eigenmodes may be defined as in [21], for example.

In some embodiments, once the desired convergence of the Electric field/threshold transmittance is obtained, no further passes are needed or formed and the iteration is stopped.

The step of Block 604 may further include selecting, determining and/or forming the number of passes (e.g., a threshold or minimum number) such that an electric field of the phase conjugate EM radiation or input EM radiation has converged to an optimal electric field for transmission of the phase conjugate EM radiation or input EM radiation through the sample. Once set criteria or thresholds are met (e.g., convergence to a pre-determined or acceptable amount from the optimal value of the electric field), iterations/repeated passes may be stopped or halted. The step may comprise forming and/or selecting the number of passes such that the EM fields have maximum transmittance through the sample.

The step of Block 604 may include selecting and/or forming a number n of the passes by comparing transmittivities λᵢ (e.g., as found in Eqn. (3), for example) of channels of the sample, or transmission of the sample, at one or more of the passes, e.g., the n^{th} pass, at each pass, or the last performed pass, as described in block 602. These steps may use detectors, for example, such as CCDs placed appropriately (e.g., the CCD in the DOPC unit).

The threshold transmittance may be such that, and/or the step may further include, selecting at least a threshold number n of the passes such that, one or more transmissions of the EM radiation through one or more most or more transmissive channels of the sample is increased by a threshold amount as compared to one or more transmissions through one or more less transmissive channels of the sample. For example, the threshold transmittance may be such that, and/or the step may comprise selecting at least a number n of the passes such that, (λ₁/λ₂)ⁿ≧5 or (λ₁/λ₂)ⁿ≧10 (and the number of passes can be increased to a finite or infinite number), wherein λ₁ is a measure of the transmittivity or transmittance of the most or more transmissive of the channels and λ₂ is a measure of the transmittivity or transmittance of the next most transmissive of the channels after a first pass of the EM radiation.

The step of block 604 may comprise selecting a number i of channels, wherein each of the channels has a transmittivity λᵢ. The number of channels may be selected by selecting an area of the sample and the channels may be defined as terminating at points on the input and output faces of the sample where an electric field or the EM radiation is measured (e.g., addressable points 708 as illustrated in FIG. 7 or speckle grains as described in [14]. The transmittivities may be eigenvalues λᵢ of a single value matrix decomposition (e.g., Eq. (3) of a transfer matrix K for the sample, for example.

The selecting or forming of a specific number of passes may not depend on the threshold transmittance (or the number of passes may not be important or may not matter) if two or three of the most transmissive channels have a transmittivity that is at least twice a mean value of all the λᵢ. The selecting or forming of a specific number of passes may not depend on the threshold transmittance (or the number of passes may not be important, or may not matter) if all the transmittivities of the channels are similar, or a group of channels have one or more transmittivities that are similar (e.g., within 5% of each other). In these situations, the electromagnetic field may already have enhanced or optimized transmission.

The step 604 may further comprise maintaining or increasing a power of the input EM radiation in one or more of the passes as compared to a power of the input EM radiation in a previous pass (e.g., but not limited to an immediately preceding pass) of the EM radiation. The maintaining or increasing of the power of the input EM radiation may suppress an effect of the irradiation resulting from one or more of the previous passes (e.g., unwanted tissue excitation or tissue damage caused by previous passes). Adjusting the power may increase or maintain the power of the input EM radiation that passes through a most transmissive channel of the sample. Increasing or maintaining the power of the input EM radiation may adjust for transmission losses in the sample. The power of the input EM radiation may be controlled to be below a threshold power that causes superficial damage to the sample/tissue.

Blocks 600-604 further illustrate forming a pass of EM radiation through the sample, the pass of EM radiation comprising (1) input EM radiation incident on the sample, and (2) transmitted EM radiation exiting the sample formed from the input EM radiation that is transmitted through the sample; (b) forming a phase conjugate of the transmitted EM radiation that is used as the input EM radiation in a next pass of the EM radiation; and (c) repeating (a) and (b) to at least form the number of passes of the EM radiation that results in one or more EM fields of the EM radiation having at least a threshold transmittance or maximum threshold scattering amount through the sample. The repeating step may include determining transmittivities or power (block 602) after one or more passes to determine if the threshold transmittance has been met and/or to adjust the power of the EM radiation in one or more of the passes (Block 604).

Block 606 represents using the EM radiation comprising the one or more EM fields having the at least a threshold transmittance or threshold scattering amount through the sample, to irradiate the sample for various applications. The step may irradiate a target within the sample, with phase conjugate or input EM radiation, wherein the phase conjugate EM or input radiation comprises a phase conjugate of the transmitted radiation in the last performed pass. The target may be preferentially irradiated with the phase conjugate EM radiation from the last pass as compared to the EM radiation from one or more, or all, previous passes. The irradiation and/or threshold transmittance may be sufficient, or tailored, for specific applications, e.g., may be used to perform photodynamic therapy on the sample, illuminate, measure, or image (e.g., a target within) the sample, or prevent or mitigate tissue or sample damage by the irradiation, for example. The EM radiation may comprise one or more optical or near infrared wavelengths, the sample may comprise tissue, and the irradiation of the target may cause therapy on or within the tissue, or image the target. In imaging applications, the power in each pass and number of passes may be adjusted or tuned to control the image contrast or focus of the target, to control imaging of the target or other object imaged on or within the sample.

The target may be more than 1 cm below a surface of the sample. When performing photodynamic therapy, the target may comprise one or more photosensitive agents or photosensitizers (e.g., chemical compounds, e.g., at a depth of more than 1 cm below a surface of the tissue), the sample may comprise tissue, and the irradiating of the photosensitive agents in step (b) may trigger therapy (e.g., reduction of a cancerous tumor or other defect) on or within the tissue or other part of an animal, human body or plant. For, example, the irradiating my reduce the tumor more effectively and with less undesired side-effects as compared to irradiating with input light from a previous (or immediately preceding pass) or as compared to photodynamic therapy that does not use the method of FIG. 6.

However, applications of the irradiation are not limited, and the electromagnetic radiation (e.g., electric field, transmission, etc.) may be tailored for a wide range of applications. The number of passes and/or threshold transmittance may be such that the electric field/transmission is sufficient to perform Block 606 more effectively.

Block 608 represents repeating one or more of the steps of block 600-606, thereby continuously re-optimizing and compensating for scatterers in the sample shifting over time. For example, after the irradiating in step 606, the EM radiation may continue through the sample to form a pass, and the EM radiation may continue to form additional passes through the sample. The step 608 may further comprise repeating steps to update the number of passes and/or maintain or increase the threshold transmittance as a function of time. The steps may be repeated until the application is halted or stopped 610, for example.

A number n of the passes and/or a power of the input EM radiation and/or the input electric field/phase in one or more of the passes, may be adjusted or tuned based on feedback received from one or more results of the irradiating step in Block 606 or irradiating by the EM field in any of the number of passes (e.g., observation of unacceptable tissue damage or insufficient activation of the photosensitive agents, inadequate tumor reduction, or imperfect imaging), or in order to deliver a prescribed or predetermined amount of power of the EM radiation to the sample and a prescribed amount of power to the target. The tuning/adjusting may deliver a dose of power, control photodynamic efficiency, excitation efficiency, while reducing/eliminating/preventing undesirable exposure (e.g., undesirable power levels) to areas of the sample where reduced or no power should be delivered (e.g., healthy tissue). Detectors (e.g., CCDs) may be used to monitor the power levels.

Steps may be added or omitted as desired.

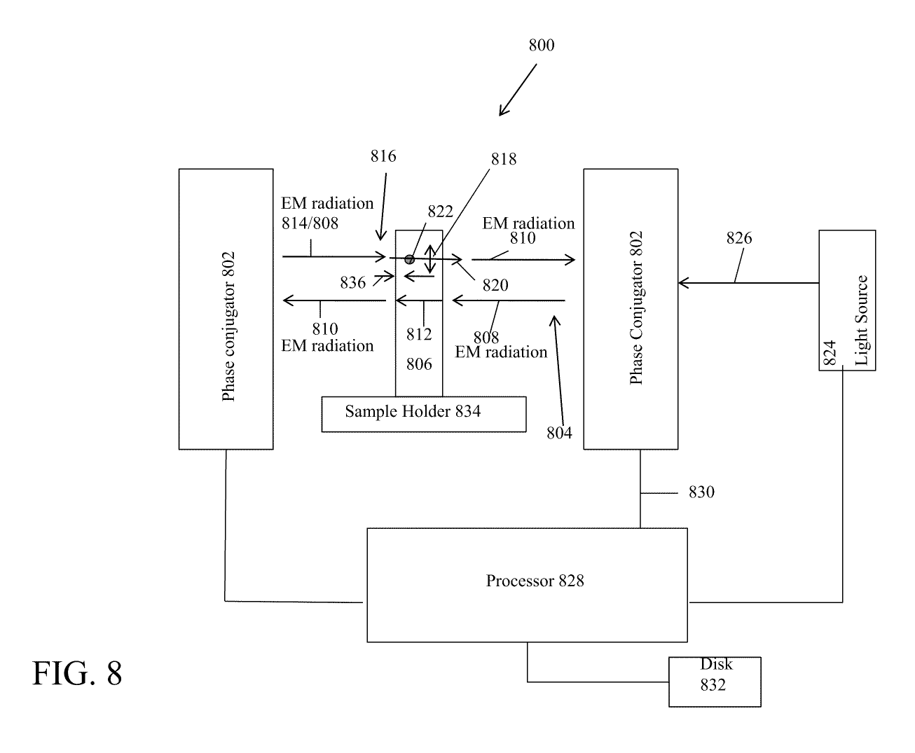

FIG. 8 illustrates an apparatus 800 for irradiating a sample with electromagnetic (EM) radiation, according to one or more embodiments of the present invention.

The apparatus 800 comprises one or more phase conjugators 802 (comprising e.g., DOPC devices, holographic material, or photorefractive crystals such as lithium niobate, for example) positioned to:
form a threshold number of passes 804 of EM radiation through a sample 806, wherein the EM radiation in each of the passes 804 comprises (1) input EM radiation 808 incident on the sample 806 and (2) transmitted EM radiation 810 exiting the sample 806 formed from the input EM radiation 808 that is transmitted 812 through the sample 806;
form a phase conjugate 814 of the transmitted EM radiation 810 from one of the passes 804, wherein the phase conjugate 814 is used as the input EM radiation 808 in a next pass 816 of the EM radiation; and
irradiate the sample 806 (e.g., a target 822 within the sample 806) with one or more EM fields 818 of the EM radiation 820, the EM fields 818 having at least a threshold transmittance or threshold scattering amount through the sample 806, the threshold transmittance or threshold scattering amount resulting from the EM radiation having made the threshold number of passes through the sample.

The apparatus 800 further comprises one or more light sources 824 coupled to the phase conjugators 802 (e.g., inputting light or EM radiation 826 into one or more of the phase conjugators 802), for performing one or more of the steps illustrated in FIG. 6, e.g., for imaging or performing photodynamic therapy, wherein the target 822 comprises one or more photosensitive agents, and the irradiating of the photosensitive agents with the phase conjugate EM radiation triggers the photodynamic therapy.

The apparatus 800 further comprises one or more processors 828 or control units coupled 830 to the phase conjugators 802 and/or light source 824, for performing one or more of the steps in FIG. 6. The apparatus may further comprise a computer readable storage medium 832 (e.g., computer disc, etc.) encoded with computer program instructions for the processor 828 to execute or perform the steps illustrated in FIG. 6.

For example, using the phase conjugator 802 that is the DOPC of FIG. 2 or FIG. 3, the processor 828/218 may control an EO modulator or other switching mechanism to switch the blank beam 326 on or off (thereby controlling the number of passes), or control a light source 824 to control the power of the blank beam 326, thereby controlling a power of the input EM radiation in one or more of the passes.

The computer readable storage medium 832 and/or processor 828 may also be used to control the number of passes to control absorption of the sample, as described in U.S. Utility patent application Ser. No. 12/886,320, filed on Sep. 20, 2010, by Zahid Yaqoob, Emily McDowell and Changhuei Yang, entitled “OPTICAL PHASE PROCESSING IN A SCATTERING MEDIUM,” attorney's docket number 176.54-US-D1.

The phase conjugators 802, DOPC, may comprise or include the processors 828, 218, e.g., as shown in FIG. 2.

FIG. 8 further illustrates a sample holder 834 configured to hold the sample that is tissue, such that the sample is optically coupled to the phase conjugators 802.

Each of the phase conjugators 802 may further comprise a detector (e.g., CCD) positioned to detect the transmitted light 810 to form a signal; and one or more spatial light modulators (SLM), coupled to the detector, to form the phase conjugate of the transmitted light 810 and to form the phase conjugated EM radiation 814, using the signal, as illustrated in FIG. 5. The power may be controlled by reflecting or outputting more light from the phase conjugator (e.g., SLM) than is received by the sensor in the phase conjugator after transmission through the sample.

The target 822 may be at a depth 836 of more than 1 cm below a surface (e.g., input face, face 1 or face 2) of the sample 806.

Hardware Environment

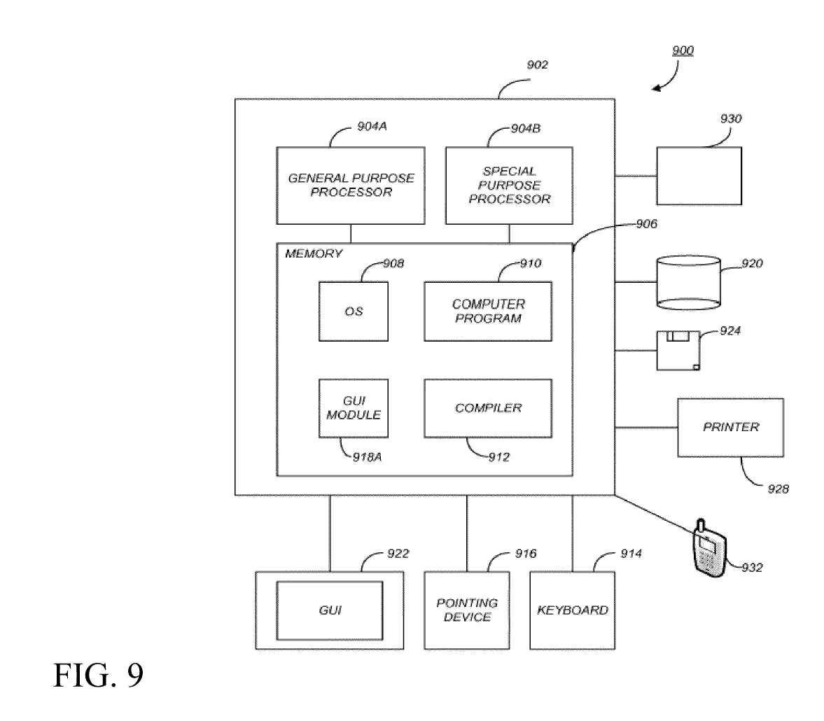

FIG. 9 is an exemplary hardware and software environment 900 that may be used in the processors 826 to implement one or more embodiments of the invention. The hardware and software environment includes a computer 902 and may include peripherals. Computer 902 may be a user/client computer, server computer, or may be a database computer. The computer 902 comprises a general purpose hardware processor 904A and/or a special purpose hardware processor 904B (hereinafter alternatively collectively referred to as processor 904) and a memory 906, such as random access memory (RAM). The computer 902 may be coupled to other devices, including input/output (I/O) devices such as a keyboard 914, a cursor control device 916 (e.g., a mouse, a pointing device, pen and tablet, etc.) and a printer 928. In one or more embodiments, computer 902 may be coupled to a media viewing/listening device 932 (e.g., an MP3 player, iPod™, Nook™, portable digital video player, cellular device, personal digital assistant, etc.). In one or more embodiments, computer 902 may be coupled to a VNA, or other devices used to measure the cavity complex valued resonant frequencies.

In one embodiment, the computer 902 operates by the general purpose processor 904A performing instructions defined by the computer program 910 under control of an operating system 908. The computer program 910 and/or the operating system 908 may be stored in the memory 906 and may interface with the user and/or other devices to accept input and commands and, based on such input and commands and the instructions defined by the computer program 910 and operating system (OS) 908 to provide output and results.

Output/results may be presented on the display 922 or provided to another device for presentation or further processing or action. In one embodiment, the display 922 comprises a liquid crystal display (LCD) having a plurality of separately addressable liquid crystals. Each liquid crystal of the display 922 changes to an opaque or translucent state to form a part of the image on the display in response to the data or information generated by the processor 904 from the application of the instructions of the computer program 910 and/or operating system 908 to the input and commands. The image may be provided through a graphical user interface (GUI) module 918A. Although the GUI module 918A is depicted as a separate module, the instructions performing the GUI functions can be resident or distributed in the operating system 908, the computer program 910, or implemented with special purpose memory and processors.

Some or all of the operations performed by the computer 902 according to the computer program 910 instructions may be implemented in a special purpose processor 904B. In this embodiment, the some or all of the computer program 910 instructions may be implemented via firmware instructions stored in a read only memory (ROM), a programmable read only memory (PROM) or flash memory within the special purpose processor 904B or in memory 906. The special purpose processor 904B may also be hardwired through circuit design to perform some or all of the operations to implement the present invention. Further, the special purpose processor 904B may be a hybrid processor, which includes dedicated circuitry for performing a subset of functions, and other circuits for performing more general functions such as responding to computer program instructions. In one embodiment, the special purpose processor is an application specific integrated circuit (ASIC).

For example, the specially programmed computer or processor 826/902/218 may receive various data from one or more detectors (e.g. CCDs), including , results from the irradiation, electric or EM field phase and amplitude at one or more points of the input and output faces of the sample at one or more passes, and power at one or more of the passes, as described in FIG. 6. Using the various data received in the specially programmed computer 828/902/218, the specially programmed computer 828 may calculate transmittivities, transmission of the sample, transmittance at one or more of the passes, to determine if the threshold transmittance has been satisfied, and/or compare transmittivities or transmittance of one or more the channels at one or more of the passes (e.g., determine if λ₁/λ₂)ⁿ≧5). The specially programmed computer may determine the threshold number of passes that obtains the threshold transmittance, and or the power needed to maintain or increase the power in each pass. Then, the specially programmed computer outputs, to the phase conjugators, the number of passes that results in one or more EM fields of the input EM radiation having at least a threshold transmittance through the sample, and/or outputs a power to the light source that maintains or increases a power of the input EM radiation in one or more of the passes.

The computer 902 may also implement a compiler 912 which allows an application program 910 written in a programming language such as COBOL, Pascal, C++, FORTRAN, or other language to be translated into processor 904 readable code. After completion, the application or computer program 910 accesses and manipulates data accepted from I/O devices and stored in the memory 906 of the computer 902 using the relationships and logic that was generated using the compiler 912.

The computer 902 also optionally comprises an external communication device such as a modem, satellite link, Ethernet card, or other device for accepting input from and providing output to other computers 902.

In one embodiment, instructions implementing the operating system 908, the computer program 910, and the compiler 912 are tangibly embodied in a computer-readable medium, e.g., data storage device 920, which could include one or more fixed or removable data storage devices, such as a zip drive, floppy disc drive 924, hard drive, CD-ROM drive, tape drive, etc. Further, the operating system 908 and the computer program 910 are comprised of computer program instructions which, when accessed, read and executed by the computer 902, causes the computer 902 to perform the steps necessary to implement and/or use the present invention or to load the program of instructions into a memory, thus creating a special purpose data structure causing the computer to operate as a specially programmed computer executing the method steps described herein. Computer program 910 and/or operating instructions may also be tangibly embodied in memory 906 and/or data communications devices 930, thereby making a computer program product or article of manufacture according to the invention. As such, the terms “article of manufacture,” “program storage device” and “computer program product” as used herein are intended to encompass a computer program accessible from any computer readable device or media.

Of course, those skilled in the art will recognize that any combination of the above components, or any number of different components, peripherals, and other devices, may be used with the computer 902.

Although the term “user computer” or “client computer” is referred to herein, it is understood that a user computer 902 may include portable devices such as cell phones, notebook computers, pocket computers, or any other device with suitable processing, communication, and input/output capability.

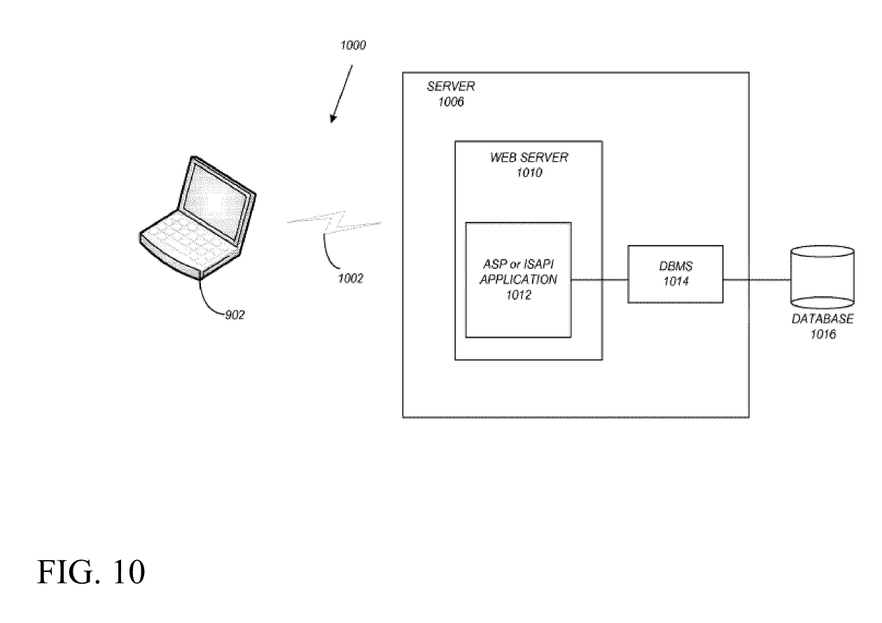

FIG. 10 schematically illustrates a typical distributed computer system 1000 using a network 1002 to connect client computers 902 to server computers 1006. A typical combination of resources may include a network 1002 comprising the Internet, LANs (local area networks), WANs (wide area networks), SNA (systems network architecture) networks, or the like, clients 902 that are personal computers or workstations, and servers 1006 that are personal computers, workstations, minicomputers, or mainframes (as set forth in FIG. 9).

A network 1002 such as the Internet connects clients 902 to server computers 1006. Network 1002 may utilize ethernet, coaxial cable, wireless communications, radio frequency (RF), etc. to connect and provide the communication between clients 902 and servers 1006. Clients 902 may execute a client application or web browser and communicate with server computers 1006 executing web servers 1010. Such a web browser is typically a program such as MICROSOFT INTERNET EXPLORER™, MOZILLA FIREFOX™, OPERA™, APPLE SAFARI™, etc. Further, the software executing on clients 902 may be downloaded from server computer 1006 to client computers 1002 and installed as a plug in or ACTIVEX™ control of a web browser. Accordingly, clients 902 may utilize ACTIVEX™ components/component object model (COM) or distributed COM (DCOM) components to provide a user interface on a display of client 902. The web server 910 is typically a program such as MICROSOFT'S INTERNENT INFORMATION SERVER™.

Web server 1010 may host an Active Server Page (ASP) or Internet Server Application Programming Interface (ISAPI) application 1012, which may be executing scripts. The scripts invoke objects that execute business logic (referred to as business objects). The business objects then manipulate data in database 1016 through a database management system (DBMS) 1014. Alternatively, database 1016 may be part of or connected directly to client 902 instead of communicating/obtaining the information from database 1016 across network 1002. When a developer encapsulates the business functionality into objects, the system may be referred to as a component object model (COM) system. Accordingly, the scripts executing on web server 1010 (and/or application 1012) invoke COM objects that implement the business logic. Further, server 1006 may utilize MICROSOFT'S™ Transaction Server (MTS) to access required data stored in database 1016 via an interface such as ADO (Active Data Objects), OLE DB (Object Linking and Embedding DataBase), or ODBC (Open DataBase Connectivity).

Generally, these components 1006-1016 all comprise logic and/or data that is embodied in/or retrievable from device, medium, signal, or carrier, e.g., a data storage device, a data communications device, a remote computer or device coupled to the computer via a network or via another data communications device, etc. Moreover, this logic and/or data, when read, executed, and/or interpreted, results in the steps necessary to implement and/or use the present invention being performed.

Although the term “user computer”, “client computer”, and/or “server computer” is referred to herein, it is understood that such computers 902 and 1006 may include portable devices such as cell phones, notebook computers, pocket computers, or any other device with suitable processing, communication, and input/output capability.

Of course, those skilled in the art will recognize that any combination of the above components, or any number of different components, peripherals, and other devices, may be used with computers 902 and 1006.

### Software Embodiments

Embodiments of the invention are implemented as a software application on a client 902 or server computer 1006.

Advantages and Improvements

The present disclosure illustrates that photodynamic therapy has a very strong potential to become a highly important, safer and more broadly applicable cancer treatment option. One or more embodiments of the present invention address its major depth penetration limitations, allowing PDT to achieve its potential.

The enhanced light delivery strategy is enabled by characterization of the time-reversal scattering suppression phenomenon [7-12].

At least one of the ways embodiments of the present invention are unique is because they directly tackle the problem of delivering more light through tissues via fundamental wavefront tailoring. Prior attempts at penetrating light deeper into tissues had focused on designing better light delivery probes for insertion into the tissues [6]. On the other hand, one or more embodiments of the present invention are non-invasive and do not involve surgery to any extent.

One or more embodiments of the present invention may benefit PDT in at least two ways. First, clinicians may now (1) deliver more light to their targets while keeping to their current incident fluence specifications for existing applications, or (2) lower the fluence and still deliver the same light dosage to their targeted sites. Second, clinicians may increase PDT treatment depth. Photodynamic therapy has excellent potential to become a highly important, safer and more broadly applicable cancer treatment option. By addressing PDT's major depth penetration limitation, as described herein, PDT may achieve this potential.

Problems/effects caused by tissue movements may also be mitigated or eliminated. The initial convergence process may be slowed down by tissue changes, but even if the initial convergence takes up to 10 seconds, it would not significantly impact the length of the entire PDT procedure (PDT treatment typically takes minutes). Once a convergence solution has been reached, one or more embodiments of the present invention may quickly track and correct for tissue changes. In fact, the response time may be ˜30 ms or less (much shorter than the live tissue dephasing time of 1.5 s measured in Ref. [18]). In addition, the OPC scattering suppression process is surprisingly robust versus errors [9].

The high number of elements used in wavefront processing may also be reduced. While ˜512×512 points on each face of the sample may be tracked, this implies the wavefront has 2.6×10⁵ elements and the involved K matrix has 7×10¹⁰ elements. One or more embodiments of the present invention, on the other hand, do not quantify K, but rather find the dominant open channel mode of K. The larger the K is, the more likely K may contain a high-transmittivity open channel mode.

The system may be optimized and one or more embodiments are not limited to the type of sample used. For example, light transmission enhancement/PDT may be performed on larger and more costly animal models, such as rabbits, or humans, and any part of the animal or human (e.g., bone, etc.).

In design, transmissive mode (transmission mode geometry) may be utilized to enable deep penetration (e.g., exceeding 6 cm through biological tissue). Thus, effective PDT treatment for certain breast cancers (by sandwiching the breast between two glass sheets), cancers in the hands, feet and oral cavity may be provided. However, the applications are not limited, and great thickness transmission may be achieved, thereby further broadening the PDT application range. Moreover, the applications are not limited to those described herein, and may include imaging for example (e.g., imaging regions deep within the tissue or turbid medium). Thus, embodiments of the present invention are not limited to enhancing transmission through tissues.

Conclusion

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

### REFERENCES

The following references are incorporated by reference herein:

[1]. Perez, C., L. Brady, E. Halperin, and R. Schmidt-Ullrich, Principles and practice of radiation oncology, 4th ed. 2003.

[2]. Dolmans, D. E. J. G. J., D. Fukumura, and R. K. Jain, Photodynamic therapy for cancer. Nat Rev Cancer, 2003. 3(5): p. 380-387.

[3]. Dougherty, T. J., C. J. Gomer, B. W. Henderson, G. Joni, D. Kessel, M. Korbelik, J. Moan, and Q. Peng, Photodynamic therapy. Journal of the National Cancer Institute, 1998. 90(12): p. 889-905.

[4]. Vo-Dinh, T., Biomedical photonics handbook. 2003, New York: CRC press.

[5]. Caimduff, F., M. R. Stringer, E. J. Hudson, D. V. Ash, and S. B. Brown, Supeicial photodynamic therapy with topical 5-ALA for supeicial primary and secondary skin-cancer. British Journal Of Cancer, 1994. 69(3): p. 605-608.

[6]. van den Bergh, H., On the evolution of some endoscopic light delivery systems for photodynamic therapy. Endoscopy, 1998. 30(4): p. 392-407.

[7]. Cui, M., E. J. McDowell, and C. H. Yang, An in vivo study of turbidity suppression by optical phase conjugation (TSOPC) on rabbit ear. Optics Express, 2009. 18(1): p. 25-30.

[8]. Cui, M., E. J. McDowell, and C. H. Yang, Observation of polarization-gate based reconstruction quality improvement during the process of turbidity suppression by optical phase conjugation. Applied Physics Letters, 2009. 95(12).

[9]. Cui, M. and C. H. Yang, Implementation of a digital optical phase conjugation system and its application to study the robustness of turbidity suppression by phase conjugation. Optics Express, 2010. 18(4): p. 3444-3455.

[10]. Tseng, S. and C. Yang, 2-D PSTD Simulation of optical phase conjugation for turbidity suppression. Optics Express, 2007. 15: p. 16055.

[11]. Yaqoob, Z., D. Psaltis, M. S. Feld, and C. Yang, Optical phase conjugation for turbidity suppression in biological samples Nature Photonics, 2008. 2: p. 110-115.

[12]. McDowell, E., M. Cui, I. Vellokoop, V. Senekerimyan, Z. Yaqoob, and C. Yang, Turbidity suppression from the ballistic to the diffusive regime in biological tissues using optical phase conjugation. Journal Of Biomedical Optics, 2010. 15: p. 025004.

[13]. Beenakker, C. W. J., Random-matrix theory of quantum transport. Reviews of Modern Physics, 1997. 69(3): p. 731.

[14]. Popoff, S. M., G. Lerosey, R. Carminati, M. Fink, A. C. Boccara, and S. Gigan, Measuring the Transmission Matrix in Optics: An Approach to the Study and Control of Light Propagation in Disordered Media. Physical Review Letters. 104(10): p. 4.

[15]. Leith, E. N. and J. Upatneiks, Holographic imagery through diffusing media. JOSA, 1966. 56: p. 523.

[16]. Yaqoob, Z., D. Psaltis, M. S. Feld, and C. Yang, Optical phase conjugation for turbidity suppression in biological samples. Nature Photonics, 2008. 2(2): p. 110-115.

[17]. McDowell, E. J., M. Cui, I. M. Vellekoop, V. Senekerimyan, Z. Yaqoob, and C. Yang, Turbidity suppression from the ballistic to the diffusive regime in biological tissues using optical phase conjugation. Journal of Biomedical Optics, 2010.

[18]. Cui, M., E. J. McDowell, and C. Yang, An in vivo study of turbidity suppression by optical phase conjugation (TSOPC) on rabbit ear. Opt. Express, 2010. 18(1): p. 25-30.

[19]. Image Transmission Through an Opaque Material Sebastien Popoff,

Geoffroy Lerosey, Mathias Fink, Albert Claude Boccara, Sylvain Gigan, arXiv:1005.0532v2 [physics optics]

[20]. Phase Control Algorithm for focusing light through turbid media, Vellekoop et. al., Optics Communications 281 (2008) p.3071.

[21] http://www.columbia.edu/itc/applied/e3101/SVD appocations.pdf

What is claimed is:

1. A method for irradiating a sample with electromagnetic (EM) radiation, comprising:
irradiating a sample with the EM radiation, wherein:
(i) the EM radiation comprises one or more EM fields having at least a threshold transmittance or threshold scattering amount through the sample,
(ii) the threshold transmittance or threshold scattering amount results from the EM radiation having made a threshold number of passes through the sample,
(iii) the EM radiation in each of the passes comprises: (1) input EM radiation incident on the sample; and (2) transmitted EM radiation exiting the sample formed from the input EM radiation that is transmitted through the sample, and
(iv) a phase conjugate of the transmitted EM radiation is used as the input EM radiation in a next pass of the EM radiation.

2. The method of claim 1, further comprising determining the number of passes such that the EM fields provide the threshold transmittance that has converged to a maximum transmittance through the sample.

3. The method of claim 1, wherein the threshold transmittance is such that a transmission of the EM radiation through one or more most transmissive channels of the sample is increased by a threshold amount as compared to a transmission through one or more less transmissive channels of the sample.

4. The method of claim 1, further comprising selecting a number n of the passes such that a transmission or transmittance of the EM radiation through the sample at the n^{th} pass does not change by more than 10% as compared to a transmission of the EM radiation through the sample in an immediately preceding pass.

5. The method of claim 1, wherein the number of passes is such that the electric fields are described by a superposition of one or more eigenmodes, the eigenmodes are eigenvectors of a singular matrix decomposition of the electric fields transmitted through the sample, and each of the eigenmodes represents one of the three most open channels of the sample.

6. The method of claim 1, wherein the number of passes is selected independent of the threshold transmittance if:
(1) two or three of the most transmissive channels have transmittivities that are at least twice a mean value of the transmittivity of all the channels, or
(2) a group of the channels have one or more transmittivities that are within 5% of each other.

7. The method of claim 1, further comprising maintaining or increasing a power of the input EM radiation in one or more of the passes as compared to a power of the input EM radiation in a previous pass of the EM radiation.

8. The method of claim 1, further comprising updating the number of passes to maintain or increase the threshold transmittance as a function of time.

9. The method of claim 1, wherein the number of passes depends on feedback from a result of the irradiating.

10. The method of claim 1, further comprising performing photodynamic therapy on the sample comprising tissue, wherein the EM fields excite one or more photosensitive agents in the tissue to trigger the photodynamic therapy.

11. The method of claim 10, wherein the photosensitive agents are at a depth of more than 1 cm below a surface of the sample, the sample is tissue positioned between a first phase conjugator and a second phase conjugator, the first phase conjugator produces the phase conjugate of the transmitted radiation from the even numbered passes, the second phase conjugator produces the phase conjugate of the transmitted radiation from the odd numbered passes, the EM radiation comprises one or more optical or near infrared wavelengths, and the number of passes are formed within a scattering time of the tissue.

12. The method of claim 1, further comprising using one or more spatial light modulators to form the phase conjugate of the transmitted light.

13. An apparatus for irradiating a sample with electromagnetic (EM) radiation, comprising one or more phase conjugators positioned to:
(a) form a threshold number of passes of EM radiation through the sample, wherein:
(i) the EM radiation in each of the passes comprises: (1) input EM radiation incident on the sample; and (2) transmitted EM radiation exiting the sample formed from the input EM radiation that is transmitted through the sample,
(ii) a phase conjugate of the transmitted EM radiation, formed by the phase conjugators, is used as the input EM radiation in a next pass of the EM radiation; and
(b) irradiate the sample with the EM radiation comprising one or more EM fields having at least a threshold transmittance or threshold scattering amount through the sample, the threshold transmittance or threshold scattering amount resulting from the EM radiation having made the threshold number of passes through the sample.

14. The apparatus of claim 13, further comprising a processor that determines the number of passes such that the EM fields provide the threshold transmittance that has converged to a maximum transmittance through the sample.

15. The apparatus of claim 13, wherein the threshold transmittance is such that a transmission of the EM radiation through one or more most transmissive channels of the sample is increased by a threshold amount as compared to a transmission through one or more less transmissive channels of the sample.

16. The apparatus of claim 13, further comprising a processor that selects a number n of the passes such that a transmission or transmittance of the EM radiation through the sample at the n^{th} pass does not change by more than 10% as compared to a transmission or transmittance of the EM radiation through the sample in an immediately preceding pass.

17. The apparatus of claim 13, wherein the phase conjugators maintain or increase a power of the input EM radiation in one or more of the passes as compared to a power of the input EM radiation in a previous pass of the EM radiation.

18. The apparatus of claim 13, further comprising one or more light sources for performing photodynamic therapy on the sample comprising tissue, wherein the EM fields excite one or more photosensitive agents in the tissue to trigger the photodynamic therapy.

19. The apparatus of claim 18, wherein the photosensitive agents are at a depth of more than 1 cm below a surface of the sample, the sample is tissue positioned between the phase conjugators including a first phase conjugator and a second phase conjugator, the first phase conjugator produces the phase conjugate of the transmitted radiation from the even numbered passes, the second phase conjugator produces the phase conjugate of the transmitted radiation from the odd numbered passes, the EM radiation comprises one or more optical or near infrared wavelengths, and the number of passes are formed within a scattering time of the tissue.

20. The apparatus of claim 13, wherein the phase conjugators comprise one or more spatial light modulators to form the phase conjugate of the transmitted light.

21. A method of performing therapy on tissue, comprising:
irradiating the tissue with phase conjugate electromagnetic (EM) radiation from a spatial light modulator.
